(19) United States
(12) Patent Application Publication
(10) Pub. No.: US 2008/0050273 A1
(43) Pub. Date: Feb. 28, 2008

(54) Biodegradable Litter Amendment Material from Agricultural Residues

(76) Inventors: Foster A. AGBLEVOR, Christiansburg, VA (US); Jactone Arogo-Ogejo, Christiansburg, VA (US); Susan W. Gay, Newport, VA (US)

Correspondence Address: LATIMER, MAYBERRY & MATTHEWS IP LAW, LLP, 13873 PARK CENTER ROAD, SUITE 122, HERNDON, VA, 20171, US

(21) Appl. No.: 11/841,048

(22) Filed: Aug. 20, 2007

Related U.S. Application Data
(60) Provisional application No. 60/823,155, filed on Aug. 22, 2006.

(51) Int. Cl. A61L 11/00; A61L 9/013

(52) U.S. Cl. 422/5; 422/122; 424/76.5

(57) ABSTRACT

The present invention relates to a biodegradable material for controlling ammonia, hydrogen sulfide, odor, and/or volatile organic compounds emissions from organic wastes. The biodegradable material in accordance with the present invention may be used to control, reduce, or prevent noxious emissions from organic wastes from, for example, animals and animal production, food and food production, pets, composting, organic fertilizer, biosolids, and potting soil mixtures to name a few. The present invention also relates to sachets, bioscrubbers, biofilters, and biomass filters comprising a biodegradable material for controlling such emissions. The present invention further relates to processes for producing and processes for using a biodegradable material to control noxious emissions from organic waste. In particular, the present invention is useful with respect to managing animal wastes, including, for example, pet, poultry, swine, dairy, horse, other livestock, other animal, and human wastes.

### CROSS-REFERENCE TO RELATED APPLICATIONS

This application relies on the disclosure and claims the benefit of the filing date of U.S. Provisional Application No. 60/823,155, filed Aug. 22, 2006, the entire disclosure of which is herein incorporated by reference.

### BACKGROUND OF THE INVENTION

1. Field of the Invention

The present invention relates to the fields of chemistry and environmental engineering. More particularly, the present invention relates to biodegradable materials for controlling noxious emissions from organic wastes, including animal waste.

2. Description of Related Art

U.S. poultry and livestock producers are increasingly concerned about emissions of ammonia (NH₃), odor, and particulate matter (PM) from their operations because: 1) the U.S. Environmental Protection Agency (EPA) is in the process of publishing regulations requiring animal feeding operations (AFOs) to comply with the applicable Clean Air Act (CAA); Comprehensive Environmental Response, Compensation and Liability Act (CERCLA); and Emergency Planning and Community Right-to-Know Act (EPCRA) provisions and 2) there has been an increase in court challenges about air quality regulations in animal agriculture. For example, CERCLA requires the operator of any facility emitting more than 45.5 kg/day (100 lbs/day) of a regulated pollutant to report such emissions.

In addition to regulatory requirements, the production and emission of NH₃ from AFOs are of concern due to potential environmental damage, and loss of fertilizer value when animal waste is applied to agricultural land. Deposition of volatilized NH₃ may cause eutrophication of surface waters, foliar damage of NH₃-sensitive plants, and soil acidification through nitrification and leaching. See H. Kirchmann, et al., “Ammonia emissions from agriculture,” Nutrient Cycling in Agro Ecosystems: 51: 1-3 (1998).

Ammonia is also an indoor air pollutant, which degrades air quality in animal production facilities. It has been reported, for example, that in poultry housing 1) exposure of birds to NH₃ increases susceptibility to respiratory diseases and 2) birds can detect and will avoid NH₃ at or below 25 ppm. See Kristensen and Wathes, “Ammonia and poultry welfare: a review,” World's Poultry Science Journal, 56:235-245 (2000). It has also been reported, that given a choice, broilers will avoid environments with NH₃ concentrations commonly found in poultry buildings. See Jones, et al., “Avoidance of ammonia by domestic fowl and the effect of early experience,” Applied Animal Behavior Science, 90:293-308 (2004).

The main source of ammonia, NH₃, in poultry housing is excreted uric acid. Ammonia is a colorless alkaline gas that is produced during decomposition of nitrogenous organic matter (i.e., matter containing nitrogen atoms, N) through bacterial deamination, or reduction of nitrogenous substances. See Kristensen and Wathes, 2000, above. In broiler housing where litter is used for bedding, the most important factors that influence NH₃ production are temperature, ventilation rate, humidity, age of litter, litter pH, litter moisture content, litter type, stocking density, and age of birds. See, e.g., A. Al-Homidan, et al., “The effect of temperature, litter and light intensity on ammonia and dust production and broiler performance,” British Poultry Science, 38: S5-S7 (1997); Yoder and van Wicklen, “Respirable aerosol generation by broiler chickens,” Transactions of the American Society of Agricultural Engineers, 31:1510-1517 (1988); and Elliot and Collins, “Factors affecting ammonia release in broiler houses,” Transactions of the American Society of Agricultural Engineers, 25:413-418 (1982).

The reduction of NH₃ concentrations inside poultry housing is important to the health of birds and workers, especially in winter when NH₃ concentrations are typically higher due to reduced ventilation rates to conserve heat. See Casey, et al., “Ammonia Emissions from Kentucky Broiler Houses during Winter, Spring and Summer,” Proceedings of A&WMA 97th Annual Conference & Exhibition: Sustainable Development: Gearing Up for the Challenge, Jun. 22-25, 2004, Pittsburgh, Pa.: A&WMA; and Wathes, et al., “Concentrations and emissions rates of aerial ammonia, nitrous oxide, methane, and carbon dioxide, dust and endotoxins in UK broiler and layer houses,” British Poultry Science, 38: 14-28 (1997).

Some methods currently being used to reduce indoor NH₃ concentrations in poultry housing include dietary manipulation to reduce excretion of nitrogen. See Robertson, et al., “Commercial scale studies of the effect of broiler protein intake on aerial pollutant emissions,” Biosystems Engineering 82(2): 217-225 (2002); and Elwinger, and Svensson, “Effect of dietary protein content, litter and drinker type on ammonia emission from broiler houses,” J. Agr. Eng'g Res. 64: 197-208 (1996).

Other methods for reducing indoor NH₃ concentrations in poultry housing include using litter amendments such as alum. See, e.g., Sims and Luka-McCafferty, “On-Farm evaluation of aluminum sulfate as a poultry litter amendment: effect of litter properties,” J. Env. Qual., 31:2066-2073 (2002); Moore et al., “Reducing phosphorus runoff and inhibiting ammonia loss from poultry manure with aluminum sulfate,” J. Env. Qual., 29:37-49 (2000); Moore et al., “Effect of chemical amendments on ammonia volatilization from poultry litter,” J. Env. Qual., 24:293-300 (1995); Worley, et al., “Reduced levels of alum to amend broiler litter,” Applied Engineering in Agriculture, 16: 441-444 (2000); and Worley, et al., “Bedding for broiler chickens: two alternative systems,” Applied Engineering in Agriculture, 15: 687-693 (1999).

Another litter amendment is Poultry Guard™. See Blake and Hess, “Sodium bisulfate as litter amendment, ANR-1208, Alabama Cooperative Extension System (2001); and McWard and Taylor, “Acidified clay litter amendment,” J. Appl. Poultry Res. 9:518-529 (2000). Poultry Litter Treatment (PLT) has additionally been used as a litter amendment. See Blake and Hess, 2001, above; Pope and Chemy, “An evaluation of the presence of pathogens on broilers raised on Poultry Litter Treatment—treated litter,” Poultry Science 79:1351-1355 (2000); Terzich et al., “Effect of Poultry Litter Treatment (PLT) on death due to ascites in broilers,” Avian Diseases 42:385-387 (1998); and Terzich et al., “Effect of Poultry Litter Treatment (PLT) on the development of respiratory tract lesions in broilers,” Avian Pathology 27: 566-569 (1998).

Still further, other methods for reducing indoor NH₃ concentrations in poultry housing focus on attempts to reduce ammonia, NH₃, in the exhaust air of mechanically ventilated poultry housing.

Aluminum sulfate (otherwise referred to as alum) is often used as a best management practice (BMP) for NH₃ control in poultry housing Moore et al., 1995, above; Moore et al., “Reducing phosphorus runoff and improving poultry production,” Poultry Sci., 78:692-698 (1999); Moore et al., 2000, above; Worley et al., 1999, above; and Worley et al., 2000, above. A disadvantage of alum is that it is a dry acid and, if ingested by chicks, can cause health problems. To prevent consumption by chicks, alum must be completely incorporated in the litter. Further, alum is not biodegradable in high concentrations; the aluminum ions are potentially phytotoxic to plants and are harmful to aquatic ecosystems. See Sims and Luka-McCafferty, 2002, above.

Moore et al. (2000) reported that NH₃ concentrations in alum treated houses were lower than 25 ppm (v), which is the presumed critical level of NH₃ for poultry. See Carlile, F. S, “Ammonia in poultry houses: a literature review,” World's Poultry Sci. J. 40:99-113 (1984). These ammonia concentrations were lower than those for untreated houses during the first 3 to 4 weeks of growth. Moore et al. (2000) cited the following reasons why alum treatment of litter should be recommended as a BMP for poultry operations: 1) alum reduces NH₃ emissions in poultry houses, which decreases the potential for health-related problems for the birds and humans working in the houses, and which decreases the environmental effects of NH₃ emitted from the house; 2) alum improves bird performance (reduced mortality, increased weight gain, and feed efficiency) and lowers fuel and electricity costs due to less need to ventilate poultry houses for NH₃ control purposes; 3) alum leads to higher litter nitrogen (N) and sulfur (S) concentrations, which leads to increased fertilizer value; 4) alum decreases soluble phosphorus (P) concentration in litter and in runoff from pasture fertilized with alum treated litter; and 5) alum reduces dissolved carbon, trace metals, and growth hormones in runoffs. A recommendation that alum should be applied after each flock at the rate of 0.2 lbs/bird was also reported. Worley et al. (2000) demonstrated that much of the economic benefits of adding alum can be achieved by adding half (0.1 lbs/bird) the recommended rates.

Sims and Luka-McCafferty (2002) studied the effects of alum on the properties of the litter and reported that adding alum decreased litter pH and the solubilities of phosphorus, arsenic, copper, and zinc. This was a desirable quality because it reduces movement of these elements into surface or shallow ground waters. Sims and Luka-McCafferty (2002) also noted that amending litter with alum increases the total and water-soluble aluminum (Al) concentration. Aluminum is potentially phytotoxic to plants and has a harmful effect on aquatic ecosystems; therefore, attention should be paid to aluminum concentration when alum amendment is used.

Composting poultry litter, animal waste, or other organic materials results in a 30% to 50% reduction in mass and produces a material with uniform nutrient composition. Composting also kills pathogen and produces a stabilized product that can be stored or land applied with little or no odor. One of the negative effects of composting animal manure is loss of N through NH₃ volatilization. Composting poultry litter has a high NH₃ volatilization potential because of high N concentrations in the litter and low C:N ratios. Up to 60% of nitrogen can be lost due to composting. See DeLaune, et al., “Effect of chemical and microbial amendments on ammonia volatilization from composting litter,” J. Env. Qual., 33:728-734 (2004); Kithome, et al., “Reducing nitrogen losses during simulated composting of poultry manure using adsorbents or chemical amendments,” J. Env. Qual., 28:194-201 (1999); and Eghball, et al., “Nutrient, carbon and mass loss during composting beef cattle feedlot manure,” J. Env. Qual., 26:189-193 (1997). DeLaune et al. (2004) reported that NH₃ volatilization during the composting of poultry litter can be reduced by amending the litter with alum. Furthermore, up to 47% of initial manure nitrogen was lost during composting even with alum amendment. Amending litter with alum did not affect the composting process; however, NH₃ volatilization could be further reduced by adding other carbon sources.

Another material that has been used as a poultry litter amendment is zeolite. See McCrory and Hobbs, “Additives to reduce ammonia and odor emissions from livestock wastes: a review,” J. Env. Qual., 30:345-355 (2001); and Amon et al., “A farm-scale study on the use of clinoptilolite zeolite and de-odorase for reducing odour and ammonia emissions from broiler houses,” Bioresource Tech. 61:229-237 (1997). Zeolites are a group of crystalline minerals consisting of aluminum and silicon derived ions that have acidic properties and large surface areas. They are extensively used as cracking catalysts in the petroleum industry, dehydration of ethanol, and for gas absorptions. In their structures, zeolites have negative charges balanced with exchangeable cations such as calcium, magnesium, sodium, potassium, and iron. These ions can be readily displaced by other substances such as heavy metals and ammonium ion. Zeolites have been used to remove NH₃ from effluent and drinking water and as animal feed additives, soil amendments, aviary floor coverings, and pet filters. Zeolites have also been used to reduce NH₃ and odor emissions from livestock wastes. Disadvantages exist, however, to using zeolites, including that: 1) zeolites are not biodegradable, so their disposal presents a new problem, 2) regeneration of zeolites requires a considerable amount of energy; and 3) zeolites are expensive and add considerable cost to poultry production.

Biofilters, bioscrubbers, and biomass filters are technologies that have been used to clean ventilation exhaust air. Biofiltration is an air treatment technology that has been used in industrial applications to reduce odor emissions. This technology has been adapted to treat odor emissions from animal housing facilities. See Classen, et al., “Design and analysis of a pilot scale biofiltration system for odorous air,” Transactions of the American Society of Agr. Engineers 43 (1): 111-118 (2000); and Nicolai and Janni, “Biofiltration media mixture ratio of wood chips and compost treating swine odors,” Water Sci. and Tech. 44:261-267 (2001). Biofilters are composed of media where microorganisms reside in the biofilm surrounding the medium particles. See Classen et al., 2000, above. Biofilters have been used to remove NH₃ emitted from animal housing at efficiencies exceeding 50%. However, removal efficiencies also depend on moisture content and media characteristics. See Kim, et al., “Comparison of organic and inorganic packing materials in the removal of ammonia gas in biofilters,” J. Hazardous Materials B72: 77-90 (2000); and McNevin and Barford, “Modeling adsorption and biological degradation of nutrients on peat,” Biochem Eng J. 2: 217-228 (1998). Biomass filters use plant or biomass material (usually chopped cornstalks or corn cobs) as filter media and clean air by impaction and retention of pollutants rather than bacterial action as in biofilters. Hoff et al. (1997) reported reductions in odor and dust levels from 43 to 90% and from 52 to 83%, respectively. See Hoff, et al., “Odor removal using biomass filters,” In 5th International Symposium on Livestock Environment, 101-108, Minneapolis, Minn., (1998). No data are available on NH₃ removal by biomass filters.

The current technology for ammonia emission control is based on zeolite, alum, sodium bisulfate, and acidified bentonite clay for absorbing or reacting with the ammonia released during the biodegradation of the animal manure. Zeolites are not biodegradable and they are expensive and therefore the amendment technology is very expensive. Alum is a dry acid and unless carefully used can cause chick mortality. Alum, sodium bisulfate, and acidified bentonite all react with the ammonia forming ammonium salts, however, the salts are not biodegradable. Thus, there is a need for an effective biodegradable litter amendment material for controlling emissions, such as ammonia and odor, from organic wastes.

### SUMMARY OF THE INVENTION

The present invention relates to a biodegradable material for controlling, reducing, or preventing ammonia, hydrogen sulfide, odor, and volatile organic compounds emissions from organic waste. The present invention is applicable to controlling such emissions from any source of organic waste or any facility where organic waste may be found, including from animals and animal production houses, food and food production houses, pets and pet waste facilities, residential garbage, urinals, biosolids, composting, organic fertilizer, and potting soil mixtures to name a few. In particular, the compounds of the present invention are useful for controlling emissions from animal houses and production facilities for poultry, swine, horse, and other livestock, as well as from household pets. Sachets, bioscrubbers, biofilters, and biomass filters comprising a biodegradable material for controlling ammonia, hydrogen sulfide, odor, and volatile organic compounds emissions are also included within the scope of the invention, as well as processes for producing and using a biodegradable material to control such emissions.

The present invention includes biodegradable compositions for controlling emissions from organic waste comprising at least one steam treated agricultural residue having an acidic pH in water. The pH of the composition slurried in water can range, for example from about 1 to about 6.

In embodiments, the compositions in accordance with the invention can be prepared from any agricultural residue, such as low-value residues, including corn-, peanut-, wood-, cotton-, soybean-, wheat-, alfalfa-, rice-, and clover-based residues. In the context of this invention, the term agricultural-based residue, including specifically named residues such as corn-based residues, refers to any agricultural product whether in whole or in part, especially low-value waste agricultural products.

In embodiments, the agricultural residue is steam treated in the presence of a reaction enhancer chosen from at least one of aluminum sulfate, ferric sulfate, ferric chloride, zinc chloride, and sulfur dioxide. The steam-treated agricultural residues, whether or not processed in the presence of a reaction enhancer, can be combined with at least one of aluminum sulfate, ferric sulfate, ferric chloride, zinc chloride, and sulfur dioxide after steam treatment. The term “at least one” in the context of this application refers to one or more and includes any combination of the recited elements.

Also included within the scope of the invention are organic waste amendment compositions comprising at least one steam treated agricultural residue chosen from corn cobs, corn fodder, corn stover, corn stalks, peanut hulls, wood chips, sawdust, cotton gin waste, soybean straw, wheat straw, alfalfa stalks, rice straw, and clover leaves. In embodiments, the organic waste amendment compositions have an acidic pH, when slurried in water, such as ranging from about 1 to about 6. Such amendment compositions can be used as an amendment material for any organic waste. For example, the amendment compositions can be used for controlling, reducing, preventing, or otherwise managing noxious emissions from any organic waste, including for example animal waste, organic fertilizer, and food waste.

In embodiments, the agricultural residues for the organic waste amendment compositions are steam treated in the presence of or are combined after steam treatment with at least one of aluminum sulfate, ferric sulfate, ferric chloride, zinc chloride, and sulfur dioxide. The compositions and organic waste amendment compositions can further comprise antimicrobials or antifungals.

Also included within the scope of the invention are sachets or filters comprising at least one steam treated agricultural residue. In embodiments, the sachets or filters comprise an agricultural residue chosen from corn cobs, corn fodder, corn stover, corn stalks, peanut hulls, wood chips, sawdust, cotton gin waste, soybean straw, wheat straw, alfalfa stalks, rice straw, and clover leaves.

Processes for preparing an organic waste amendment composition according to the invention are also included. Such processes can comprise: 1) reacting at least one agricultural residue; 2) with steam under pressure; 3) for a sufficient time and temperature; and 4) releasing the pressure to obtain a composition, which has an acidic pH in water. The pH of such compositions prepared by this steam-treatment process can have a pH ranging from about 1 to about 6, such as for example a pH of from about 1 to a pH of from about 2. In embodiments, the agricultural residues are chosen from agricultural, wood, and forest waste products, such as for example corn cobs, corn fodder, corn stover, corn stalks, peanut hulls, wood chips, sawdust, cotton gin waste, soybean straw, wheat straw, alfalfa stalks, rice straw, and clover leaves, or combinations thereof.

The processes according to the invention can be performed with agricultural residues in the presence of at least one reaction enhancer chosen from aluminum sulfate, ferric sulfate, ferric chloride, zinc chloride, and sulfur dioxide.

Processes for controlling, reducing, preventing, or otherwise managing the emission of ammonia, hydrogen sulfide, odor, or volatile organic compounds from organic waste or an environment subject to organic waste are also included within the scope of the invention. Such processes may comprise providing at least one steam treated agricultural residue chosen from corn cobs, corn fodder, corn stover, corn stalks, peanut hulls, wood chips, sawdust, cotton gin waste, soybean straw, wheat straw, alfalfa stalks, rice straw, and clover leaves in an amount sufficient to control at least one of the emission(s). The term “providing” in the context of this invention refers to applying, mixing, exposing, composting, or contacting at least one steam treated agricultural residue to or with organic waste or an environment subject to organic waste, including making the compositions available for applying, mixing, exposing, etc.

### BRIEF DESCRIPTION OF THE DRAWINGS

FIG. 1 shows representative ammonia absorption capacity of SECC.

FIG. 2 shows the effect of reaction time on the ammonia absorption capacity of steam treated agricultural residues (SECC).

FIG. 3 shows the effect of reaction temperature on the ammonia absorption capacity of steam treated agricultural residues (SECC).

FIG. 4 shows the effect of a reaction enhancer (FeSO₄) on the ammonia absorption capacity of steam treated agricultural residues (SECC).

### DETAILED DESCRIPTION OF VARIOUS EMBODIMENTS OF THE INVENTION

The present invention is based on processed biodegradable plant materials, including wood, agricultural residues, and forest residues, that convert ammonia into organic acid salts such as ammonium acetate and ammonium propionate. Volatile organic compounds (VOCs) generated during decomposition of organic waste, such as manure, also react with the new material. Alcohol components of the VOCs react with the organic acids forming esters that have a sweet fruity smell. Foul odor from any organic waste can be reduced and/or masked with the ester products produced from reaction of organic waste with the inventive material.

In particular, the waste amendment compositions can be applied to any organic waste, including for example any animal waste, such as from poultry, swine, horse, or other livestock, as well as from human urine and household pets; any organic fertilizer, including potting soil mixtures; and any food waste, including wastes from food production facilities, household garbage, and/or that found in composting.

The steam-treated biodegradable material can be used as an amendment to any organic waste, including for example animal wastes, such as poultry litter. In the context of this invention, the term “amendment” refers to the compositions of the invention, which can be applied to, combined with, mixed with, exposed to, or otherwise contacted with organic wastes. The amount of material used depends on the particular application and can be equal to, exceed, or be less than the amount or organic waste being treated. The amendment material can be combined with other materials used in the management of organic waste, such as bedding or floor covering, or other materials for reducing or controlling noxious emissions from organic waste.

In organic wastes treated with the biodegradable material, inherent nitrogen can be conserved and the nitrogen content can be increased, thus, considerably enhancing the compost value of the organic wastes, in particular animal wastes. The amended material can be land applied or composted because of the increased carbon to nitrogen (C:N) ratio.

In accordance with the present invention, the biodegradable material can be prepared from any plant material. From a cost standpoint, of special interest are low-value agricultural residues, including wood and forest residues. Low-value agricultural residues include agricultural, wood, and/or forest residues or waste products, such as any part of an agricultural product that might otherwise be discarded. Low-value agricultural residues include corn wastes, for example, corn cobs, corn fodder, corn stover, and corn stalks; peanut wastes, for example, peanut hulls; wood wastes, for example, wood chips, and sawdust; and cotton wastes, for example, wastes from cotton gins; soybean residues, for example soybean straw; wheat harvest residues, for example wheat straw; alfalfa wastes, for example alfalfa stalks; rice harvest residues, for example rice straw; and clover harvest residues, for example clover leaves.

In the past, corn cobs and wood chips have been used as absorbents in the field of controlling animal wastes from, for example, animal production facilities without advance steam treatment of those materials. Such materials, however, do not absorb ammonia and other odoriferous compounds in significant quantities because they have small surface areas and do not have acidic sites. The inventors, however, have found that steam treatment of these agricultural-based materials increases their surface areas and above all, the steam-treatment process acidifies their surfaces because of the production of organic acids during the treatment process.

The acid production during the steam-treatment process is due to the decomposition of the acetyl groups in the plant material which further catalyses the decomposition of the constitutive biopolymers. The acidity of the materials are further enhanced by conducting steam explosion in the presence of sulfur dioxide or inorganic salts, such as aluminum sulfate, ferric chloride, zinc chloride, and ferric sulfate. Inorganic salts and sulfur dioxide enhance the degradation of the plant material and increase the surface area as well.

By using low-value wood, forest, and/or agricultural residues, the new material can be produced several times cheaper than zeolites or alum. Because the new materials are also biodegradable, their disposal is relatively easy. In contrast, the old technology produces inorganic salts that are not biodegradable.

The biodegradable material contains weak organic acids, such as acetic acid, propionic acid, and levulinic acids that are generated in the agricultural fiber during the process and are not known to pose any health risk to the birds or animals when exposed to the material or used as a litter amendment. The moisture content of the inventive material is relatively low (less than 20 wt %) and therefore will not create a more humid atmosphere in animal houses, such as poultry houses.

One aspect of the invention involves the conversion of agricultural residues, including wood and forest residues, such as corn cobs, corn fodder, corn stover, corn stalks, cotton gin wastes, peanut hulls, soybean straw, rice straw, and wood wastes to acidic substrates using steam explosion at various treatment severities. Steam penetrates the cells of the agricultural residues and, upon release of the steam pressure, decompression and mechanical disintegration of the agricultural residues results, which provides for a steam treated material having a large surface area and a pH in the range of about 2.4 to about 6.4. The pH can be further enhanced (made more acidic) by performing the reaction in the presence of reaction enhancers, including sulfur dioxide and/or inorganic salts.

Effectiveness of the steam treated material depends on the pH, temperature, and inorganic salts, which can be controlled by the severity of the steam explosion treatment. By optimizing the severity parameter of the steam treatment with respect to different plant materials, one skilled in the art can optimize the performance of the steam treated material in a particular application.

Another aspect of the invention involves performing the reaction in the presence of a reaction enhancer. Inorganic salts, such as aluminum sulfate (Al₂(SO₄)₃), ferric sulfate (Fe₂(SO₄)₃), ferric chloride (FeCl₃), zinc chloride (ZnCl₂), and sulfur dioxide (SO₂) in an amount ranging from 1 wt % to 10 wt % can be added and/or mixed with the plant material, for example, before or after the steam explosion process. The addition of these salts or SO₂ to the plant materials lowers the steam explosion temperature, residence time in the reactor, and increases the acidity of the steam treated material. In the presence of at least one of these reaction enhancers, the surface area of the plant material is considerably increased and the pH of the slurry prepared with water is reduced to about 1 to 2. The final pH of the steam treated material depends on which additive was added to the plant material. By optimizing the salt or SO₂ content and the severity parameter of the steam treatment with respect to the plant material, one skilled in the art can optimize the performance of the steam treated material in a particular application, for example, by preparing a material capable of absorbing noxious emissions relatively quickly or over extended periods of time.

As used herein, the biodegradable material is often referred to as AMOSOAK. Amosoak can be used as a biodegradable poultry litter amendment in poultry houses to control (for example, reduce, manage, mask, and/or prevent) emissions from animal wastes, such as by controlling, reducing, or preventing ammonia release, odor, hydrogen sulfide, and VOC emissions. Thus, the health of the birds and workers can be improved. Amosoak can also be used as a filter element in mechanical exhaust systems in poultry and animal housing to reduce emission of VOCs, ammonia (NH₃), and hydrogen sulfide, as well as other noxious emissions. Because Amosoak sequesters ammonia in the form of organic ammonium salts, it increases the nutrient value of the litter. This biodegradable material can also reduce energy costs by reducing ventilation needs.

This invention can be used in any application where controlling, reducing, preventing, or otherwise managing noxious emissions from any organic waste is desired. For example, the present invention can be used in the animal production industry and associated animal production facilities and in the food processing industry and associated food processing facilities. Of particular interest is the use of the present invention in animal production facilities, such as the poultry, swine, dairy, horse, and other animal production facilities, for ammonia emission control, odor reduction, hydrogen sulfide emission control, and VOC reduction. The biodegradable material could also be used to control ammonia, odor, hydrogen sulfide, and VOC emission from any animal waste, including pet waste and biosolids. It could also find applications in pet filters, sachets, trash cans and liners, urinals, organic fertilizer, food waste, exhaust air filters, and the chemical industries for ammonia scrubbing.

This steam treated material has high capacity to react with gaseous ammonia, reduce odor, reduce hydrogen sulfide, and reduce volatile organic compounds (VOC) emission. This aspect of the invention is different from existing technologies, in that, apart from reducing ammonia, hydrogen sulfide, and odor, it also reduces the release of volatile organic compounds that are regulated under CAA, CERCLA, and EPCRA. Above all, Amosoak is biodegradable, whereas current technologies are based on inorganic materials that are not biodegradable. For example, current litter amendment technologies, such as zeolite, alum, poultry guard (an acidified bentonite), and poultry litter treatment (PLT), are not biodegradable. Clearly, the present technology provides advantages over existing technologies.

Reference will now be made in detail to various exemplary embodiments of the invention. The following detailed description is provided to give the reader a better understanding of certain details of the aspects of the invention and should not be understood as a limitation of the invention.

### EXAMPLE I

About 1 kg of “as received” corn cob (unprocessed, e.g., not milled) was loaded into a 25-L steam explosion gun and steam was admitted into the chamber. The temperature of the corn cob was raised to 210° C. After the reaction proceeded for 60 seconds, the steam pressure was released, resulting in the decompression and mechanical disintegration of the corn cob. The steam exploded corn cob (SECC) was a fine brown powder with low moisture content (40 wt %) and, when slurried with water, had a pH of 3.65.

In a first instance, a packed-column reactor consisting of a 61×5 cm glass cylindrical vessel with a fritted glass bottom was used to evaluate the ammonia absorption capacity of the SECC. The reactor was packed with 8-cm thick SECC material and nitrogen gas containing 150 ppm of ammonia was admitted into the reactor chamber through the fitted glass distributor. The gas was passed through the 8-cm thick SECC layer for 10, 20, and 30 minute periods. The gas exiting the reactor was passed through a 2 M HCl bath. In addition, a control experiment was carried out with no SECC material in the reactor.

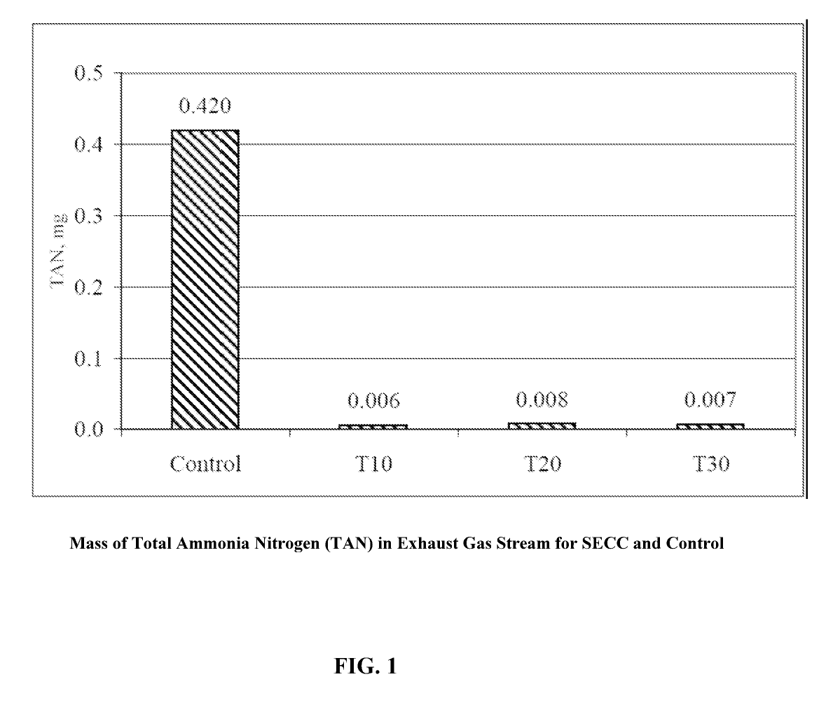

Analysis of the HCl baths showed a 98% reduction in the ammonia concentrations in the nitrogen/ammonia mixture after passing through the bed of SECC (FIG. 1). More particularly, FIG. 1 shows representative absorption of ammonia by agricultural material steam treated in accordance with the invention. In particular, the mass of total ammonia nitrogen (TAN) in exhaust gas stream collected after various flow times is shown. The values for the Control show the mass of TAN collected for 10 min. with no SECC media. T10 is the mass of TAN collected between 0 and 10 min with SECC media, T20 is the mass of TAN collected between 10 and 20 min. with SECC media; and T30 is the mass of TAN collected between 20 and 30 min. with SECC media.

The parameters of the steam treatment process can be adjusted so as to obtain compositions that absorb emissions at different rates. For example, reaction parameters, such as reaction time, reaction temperature, and whether reaction enhancers are used, can be adjusted to obtain compositions that absorb ammonia and other noxious emissions either relatively quickly or over extended periods of time. Further, compositions resulting from treatment processes operated under different conditions can be combined to obtain compositions that absorb noxious emissions both relatively quickly and over extended periods of time.

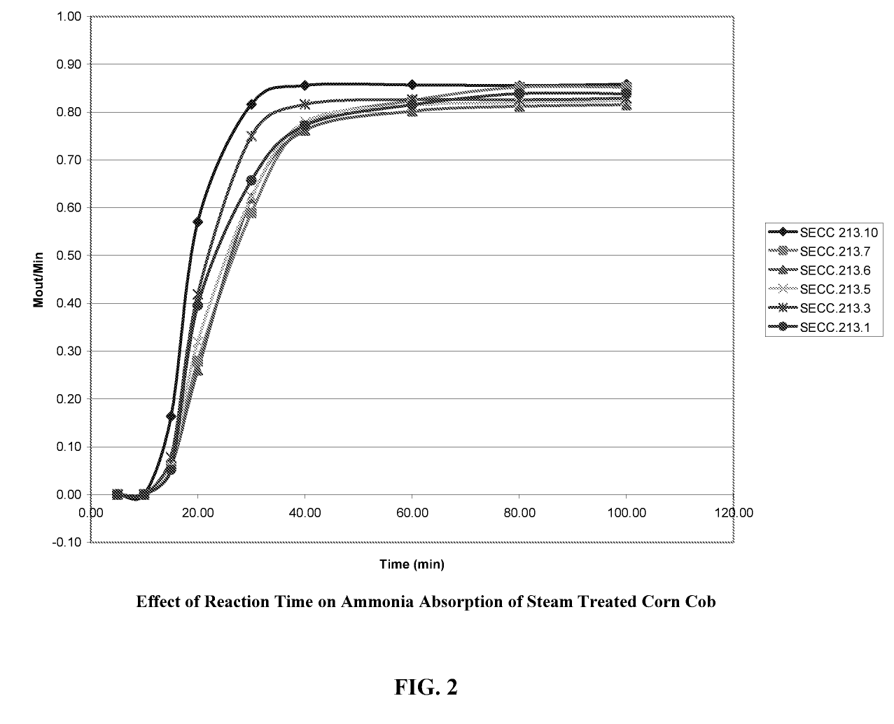

FIG. 2, for example, shows the effect of reaction time on the ammonia absorption capacity of steam exploded corn cob. As shown, corn cob was exploded at a temperature of 213° C. for various periods of time: SECC 213.10 (10 min.), SECC 213.7 (7 min.), SECC 213.6 (6 min.), SECC 213.5 (5 min.), SECC 213.3 (3 min.), and SECC 213.1 (1 min.). The maximum ammonia absorption capacity of the steam treated material leveled off at about 80-90% for each of the trials. SECC steam treated for 10 min. and 3 min. reached their maximum absorption capacity slightly faster than the remainder of the trials after about 30 min. and 40 min., respectively, following exposure to the ammonia. SECC steam treated for 1 min., 5 min., 6 min., and 7 min. reached their maximum absorption capacity between 60-70 min. following exposure to the ammonia.

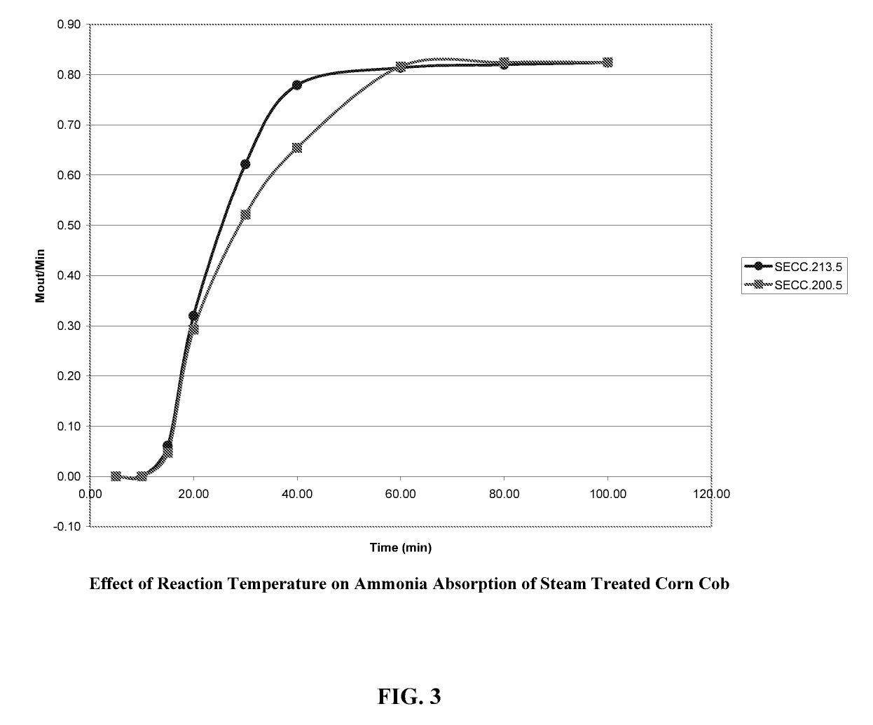

FIG. 3 shows the effect of reaction temperature on the ammonia absorption capacity of steam exploded corn cob. More particularly, as shown in FIG. 3, corn cob material was steam treated for 5 minutes at different temperatures: 200° C. and 213° C., respectively labeled as SECC 200.5 and SECC 213.5. As shown, the corn cob subjected to the higher temperature, 213° C., reached its maximum absorption capacity after about 40 min. following exposure to the ammonia, while the corn cob subjected to the lower temperature, 200° C., reached its maximum absorption capacity after about 60 min. following exposure to the ammonia.

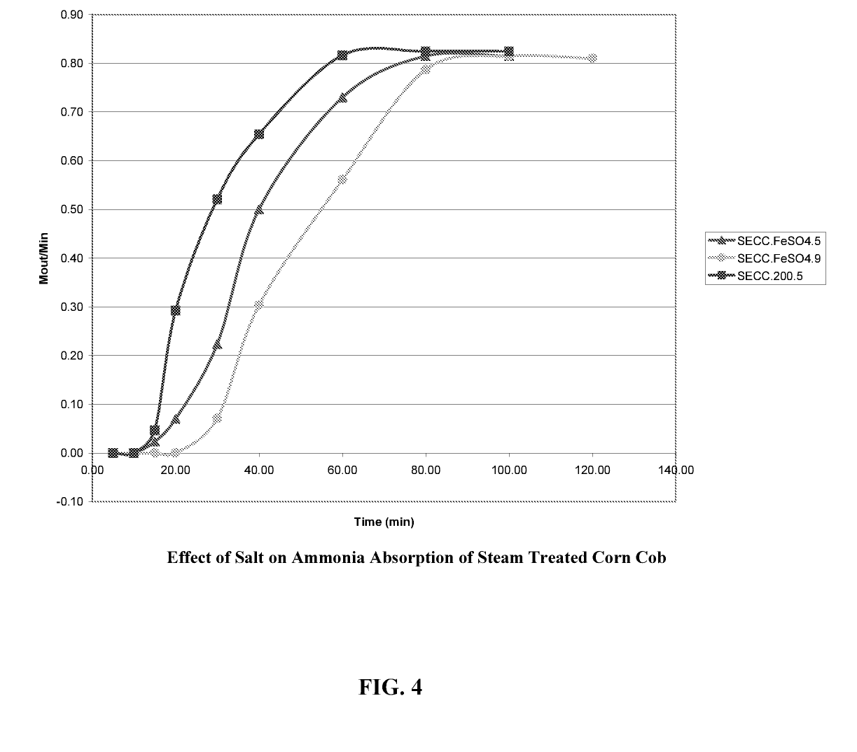

FIG. 4 shows the effect of a reaction enhancer, which is present during steam treatment, on the ammonia absorption capacity of the resulting material. More particularly, shown in FIG. 4 is a comparison of the ammonia absorption capacity of steam treated corn cob material reacted for 5 min. at 200° C., either in the presence of a reaction enhancer or without: SECC 200.5 (without ferric sulfate), SECC FeSO4.5 (with the presence of 5 wt % ferric sulfate), and SECC FeSO4.9 (with 9 wt % ferric sulfate). As shown, in general, the higher concentration of ferric sulfate present during the reaction process leads to compositions that absorb ammonia at a slower rate than compared to reactions performed in the presence of ferric sulfate at lower concentrations or reactions performed without the reaction enhancer. Although each of the compositions absorbed relatively the same amount of ammonia overall, 80-85%, each absorbed ammonia at a different rate. For example, it took about 20 min. for the SECC without any ferric sulfate present during the reaction to absorb 30% of the ammonia, while it took approximately 30 min. for the SECC with 5 wt % ferric sulfate present and approximately 40 min. for the SECC with 9 wt % ferric sulfate present to absorb the same amount.

In a second instance, steam exploded corn cob (SECC) was mixed with broiler litter in 1:1, 2:1, 3:1, 5:1, 10:1, 20:1, and 51:1 ratios of broiler litter: SECC. In addition, a control of untreated broiler litter was prepared with broiler litter and no SECC. The mixtures and the control were stored overnight in sealed “glad freezer bags.” When the bags were opened the next day, the untreated broiler litter had a strong ammoniacal smell that was very unpleasant. Treated samples with a broiler litter:SECC ratio of 1:1, 2:1, 3:1, and 5:1, however, had sweet smells typical of ester compounds, which were not offensive. The degree of sweetness appeared to correspond to the quantity of SECC mixed with the broiler litter. The treated sample with a broiler litter:SECC ratio of 1:1 produced the most pleasant smell. Treated samples with a broiler litter:SECC ratio of 10:1, 20:1, and 51:1 had slight ammoniacal smell and no sweet pleasant smell. After four weeks of storage, treated samples with a broiler litter:SECC ratio of 1:1, 2:1, 3:1, and 5:1 still had pleasant smells.

In a third instance, steam exploded corn cob (SECC) was added to putrefied chicken in a weight ratio 9:1 (putrefied chicken to SECC ratio or 10 wt %) and stored in sample bottle for three weeks. A control sample of putrefied chicken without addition of SECC was stored under same conditions for comparison. After three weeks storage, the control sample had a strong putrid odor whereas the sample with 10 wt % SECC did not have any odor and it had a pleasant smell.

In a fourth instance, steam exploded corn cob (SECC) was added to organic fertilizer in a weight ratio of 9:1 (organic fertilizer to SECC ratio or 10 wt %) and stored for three weeks. The strong odor of the organic fertilizer disappeared as soon as the SECC was mixed with the organic fertilizer. After three weeks storage, the organic fertilizer treated with SECC had no odor whereas the control sample with no SECC addition had a very strong offensive odor.

### EXAMPLE II

About 1 kg of “as received” cotton gin waste was loaded into a 25-L steam explosion gun and steam was admitted into the chamber. The temperature of the cotton gin waste was raised to 210° C. After the reaction proceeded for 60 seconds, the steam pressure was released, resulting in the decompression and mechanical disintegration of the cotton gin waste. The steam exploded cotton gin waste (SECGW) was a fine brown fibrous mixture with low moisture content (40 wt %) and, when slurried with water, had a pH of 6.0. The SECGW removed ammonia when it was packed into the cylindrical glass reactor. It also removed odor and VOC when contacted with broiler litter.

### EXAMPLE III

About 1 kg hardwood waste chips (one inch particle size) were loaded into a 25-L steam explosion gun. Steam was admitted into the chamber. The temperature of the wood chips was raised to 235° C. After the reaction proceeded for 120 seconds, the steam pressure was released, resulting in the decompression and mechanical disintegration of the wood chips. The steam exploded wood chips (SEWC) was a fine brown powder with low moisture content (40 wt %) and, when slurried with water, had a pH of 3.5. The steam exploded wood chips also showed strong reaction with ammonia, reduced odor, and VOC.

The broiler litter and broiler litter amended with SECC were subjected to head space solid phase micro-extraction (HS-SPME) for volatile compounds. In this process, the samples were placed in 20 mL head space vials. The extraction of the head space was conducted with 30/50 μm divinylbenzene/carboxene/polydimethylsiloxane SPME fiber and conducted at 60° C. for 30 minutes. The sample was desorbed for 5 minutes into the injector of a Shimadzu 2010s quadrupole mass analyzer. Separation was achieved on a SPB-1 SULFER capillary column (30 m×0.32 mm×4.0 μm film thickness) at a flowrate of 1.43 mL/minute helium. The column was held at 40° C. for 3 minutes and raised to 280° C. at 5° C./min and held at 280° C. for an additional 10 min. The results of the analysis are shown in Table 1. It is clear from Table 1, that SECC removed both volatile and odoriferous compounds. Additionally it was shown to remove methyl mercaptan from cat urine.

TABLE 1
Head space analysis data for broiler litter and Amosoak
treated broiler litter.

| | Wake broiler litter | Amosoak treated |
|---|---|---|
| Volatile compound | (relative areas) | broiler litter |
| Ethanol | 79,106 | nd |
| N,N-dimethyl-methylamine | 813,919 | nd |
| 1,2-benzenedicarboxaldehyde | 384,404 | 166,432 |
| Carbanic acid phenyl ester | 2,643,530 | nd |
| Methyl phenol | 157538 | nd |
| 2-ethylnyl pyridine | 121,443 | nd |

nd = not detected.

### EXAMPLE IV

About 1 kg of “as received” peanut hulls were loaded into a 25-L steam explosion gun. Steam was admitted into the chamber. The temperature of the peanut hulls chips was raised to 235° C. After the reaction proceeded for 120 seconds, the steam pressure was released, resulting in the decompression and mechanical disintegration of the peanut hulls. The steam exploded peanut hull (SEPH) was a fine brown powder with low moisture content (40 wt %) and, when slurried with water, had a pH of 3.0. The steam exploded peanut hull showed strong reaction with ammonia, reduced odor, and VOC.

### EXAMPLE V

The steam explosion process was scaled-up to 1000 kg/h in a continuous Stake steam exploder. Corn cobs were used as feedstock, and the material was treated with steam at 210° C. and 60 seconds and then decompressed and exploded as described for the 25-L gun. About 1000 kg of “as received” corn cob was processed in the continuous steam exploder. The product was a fine brown powder similar to the corn cob treated in the 25-L steam gun. This large-scale SECC was equally effective for ammonia, odor, and VOC removal. The results were identical to those shown in FIG. 1. When the SECC was applied to broiler litter, the results were identical to those obtained for the small-scale steam exploder. Thus, we have demonstrated that the process can be scaled-up to a capacity of 1000 kg per hour and the product is as effective as the small-scale product.

### EXAMPLE VI

About 1 kg of corn cobs was thoroughly mixed with 10, 20, and 100 g of alum and 500 g distilled water before being loaded into the 25-L steam explosion gun. The valves were closed and steam was admitted into the chamber. The biomass temperature was raised to 210° C. and the residence time for the reaction was 60 seconds. The pressure was released to the atmosphere and the material was exploded. The addition of the alum caused a more extensive degradation of the corn cob. The new material after the steam treatment had a finer texture than the samples without alum treatment. The pH of the sample slurry was 2.4 compared to 3.65 for the corn cob without the alum. When ammonia was passed through this material, this also showed a strong reaction with ammonia. When this material was added to the broiler liter at 10 wt % of the broiler litter, the ammonia, odor, and VOCs were removed.

Further, 10 wt % ferric sulfate (Fe₂(SO₄)₃) was added to steam treated corn cobs and thoroughly mixed. The addition of the ferric sulfate to the steam exploded corn cob changed the color of the material immediately from brown to dark brown. This mixture had a pH of 2.0 and was able to remove ammonia, odor, and VOC from broiler litter.

It will be apparent to those skilled in the art that additives, such as antimicrobials and/or antifungals, may be included in the compositions of the present invention in order to limit multiplication of bacteria and fungi, for example, in an animal waste amendment.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

1. A biodegradable composition for controlling emissions from organic waste comprising at least one steam treated agricultural residue of acidic pH in water.

2. The composition according to claim 1, wherein said pH of said composition in water ranges from a pH of about 1 to a pH of about 6.

3. The composition according to claim 1, wherein said at least one steam treated agricultural residue is chosen from corn-, peanut-, wood-, cotton-, soybean-, wheat-, alfalfa-, rice-, and clover-based residues.

4. The composition according to claim 3, wherein said at least one steam treated agricultural residue is steam treated in the presence of at least one of aluminum sulfate, ferric sulfate, ferric chloride, zinc chloride, and sulfur dioxide.

5. An organic waste amendment composition comprising at least one steam treated agricultural residue chosen from corn cobs, corn fodder, corn stover, corn stalks, peanut hulls, wood chips, sawdust, cotton gin waste, soybean straw, wheat straw, alfalfa stalks, rice straw, and clover leaves.

6. The organic waste amendment composition according to claim 5, wherein the pH of said composition in water is acidic.

7. The organic waste amendment composition according to claim 6, wherein said pH ranges from about a pH of 2.4 to about a pH of 6.4.

8. The organic waste amendment composition according to claim 5, further comprising ferric sulfate.

9. The organic waste amendment composition according to claim 5, further comprising at least one antimicrobial or antifungal.

10. A filter or sachet comprising at least one steam treated agricultural residue.

11. The filter or sachet according to claim 10, wherein said agricultural residue is chosen from corn cobs, corn fodder, corn stover, corn stalks, peanut hulls, wood chips, sawdust, cotton gin waste, soybean straw, wheat straw, alfalfa stalks, rice straw, and clover leaves.

12. A process for preparing an organic waste composition comprising:
reacting at least one agricultural residue,
with steam under pressure, for a sufficient time and temperature, and
releasing said pressure to obtain a composition having an acidic pH in water.

13. The process according to claim 12, wherein said pH ranges from about 1 to about 6.

14. The process according to claim 12, wherein said at least one agricultural residue is chosen from agricultural, wood, and forest waste products.

15. The process according to claim 14, wherein said at least one agricultural residue is chosen from corn cobs, corn fodder, corn stover, corn stalks, peanut hulls, wood chips, sawdust, cotton gin waste, soybean straw, wheat straw, alfalfa stalks, rice straw, and clover leaves.

16. The process according to claim 12 further comprising reacting said agricultural residue in the presence of at least one of aluminum sulfate, ferric sulfate, ferric chloride, zinc chloride, and sulfur dioxide.

17. The process according to claim 16, wherein said composition has a pH ranging from about 1 to about 2.

18. A process for controlling emission of ammonia, odor, or volatile organic compounds from organic waste or an environment subject to organic waste, comprising:
providing at least one steam treated agricultural residue chosen from corn cobs, corn fodder, corn stover, corn stalks, peanut hulls, wood chips, sawdust, cotton gin waste, soybean straw, wheat straw, alfalfa stalks, rice straw, and clover leaves in an amount sufficient to control said emission.

19. The process according to claim 18, wherein said providing comprises applying, mixing, exposing, composting, or contacting said at least one steam treated agricultural residue to or with organic waste or an environment subject to organic waste.
